(19) United States
(12) Patent Application Publication
(10) Pub. No.: US 2023/0073033 A1
(43) Pub. Date: Mar. 9, 2023

(54) PISTON STOP FOR A MEDICAL SYRINGE AND MEDICAL SYRINGE WITH A PISTON STOP

(71) Applicant: Fresenius Kabi Deutschland GmbH, Bad Homburg (DE)

(72) Inventors: Torsten Brandenburger, Reichelsheim (DE); Michael Schönhofen, Saarlouis (DE)

(21) Appl. No.: 17/759,758

(22) PCT Filed: Apr. 22, 2021
(86) PCT No.: PCT/EP2021/060471

§ 371 Date: Jul. 29, 2022

(30) Foreign Application Priority Data
Apr. 24, 2020 (EP) 20171386

(51) Int. Cl. A61M 5/315

(52) U.S. Cl. CPC A61M 5/31513; A61M 2205/0216

(57) ABSTRACT

The invention relates to a piston stopper for a medical syringe. The piston stopper has an end wall, a side wall and an internal thread for securing a plunger rod, wherein the piston stopper is made of a butyl rubber. The end wall is conical in the distal direction both on the outside and on the inside. The end wall merges towards the side wall into a first sealing ring which extends around the circumference of the side wall. The side wall has at least three further sealing rings. Further, the invention relates to a medical syringe provided with the plunger stopper.

### FIELD OF THE INVENTION

The invention relates to a plunger stopper for a medical syringe as well as a medical syringe which is equipped with the plunger stopper. In particular, the invention relates to a medical syringe packaged in a sterile manner, which is pre-filled with a medical fluid containing a medication.

### BACKGROUND OF THE INVENTION

At least in the case of pre-filled medical syringes, plungers are used in practice which comprise a plunger stopper attached to the plunger rod.

The plunger stopper consists of a material which has a higher elasticity than the material of the plunger rod.

Thus, an improved sealing effect can be achieved with simultaneously easier sliding of the plunger.

In particular in the case of syringes containing pharmaceutical products, the requirements for such a plunger stopper are high. The material used should, as far as possible, have no interactions with the filled medical fluid during storage. Furthermore, the force effort to push the plunger should be kept as low as possible. The seal-tightness increases with increasing compression of the plunger stopper in the syringe body, but the required forces to push the plunger increase at the same time. Thus, a compromise always has to be selected between seal-tightness and sliding force.

What is problematic is in particular when, due to the differences in frictional forces repeatedly occurring in practice around the circumference of the plunger, there may result a tilting of the plunger which notably increases the force required to push the plunger further.

### OBJECT OF THE INVENTION

Therefore, the object underlying the invention is to provide a plunger stopper optimally suited for a syringe pre-filled with a medical fluid.

In particular, the tendency of the plunger to tilt in the case of inhomogeneous frictional forces around its circumference should be reduced.

### SUMMARY OF THE INVENTION

The object of the invention is already achieved by a plunger stopper according to claim 1 and by a medical syringe equipped with the plunger stopper.

Preferred embodiments and further developments can be inferred from the subject matter of the dependent claims, the description and the drawings.

The invention relates to a plunger stopper for a medical syringe, wherein the plunger stopper has an end wall, a side wall and an internal thread for securing a plunger rod, wherein the plunger stopper is made of a butyl rubber, wherein the end wall is conical in the distal direction both on the outside and on the inside, wherein the end wall merges towards the side wall into a first sealing ring, which extends around the circumference of the side wall, and wherein the side wall has at least three further sealing rings.

It has been found that through the combination according to the invention of different measures, a plunger stopper can be provided which is optimally suited for a pre-filled medical syringe, in particular has a low interaction with filled medical fluids and tends to tilt only to a small extent.

The internal thread serves to exactly guide the plunger. Low interactions with medical fluids are ensured by the butyl rubber that is used.

The end wall is conical in the distal direction, i.e. in the direction of the inner volume of the syringe, when used as intended both on the outside and on the inside. In conjunction with a plunger rod with a conical end, which engages into the plunger stopper, the tension caused by frictional forces is concentrated in the material at the tip of the plunger stopper.

The deforming material therefore returns to its original shape with changing frictional forces more so in the direction of the tip and thus has less tendency to tilt to the side.

The end wall is preferably a truncated cone shape. The upper side of the end wall merges continuously into a first sealing ring upon reaching the side wall.

The side wall begins behind the end wall, i.e. directly with a sealing ring, which extends around its circumference. Thus, there is no cylindrical side wall section provided between the edge of the end wall and the side wall.

At least three further sealing rings extend from this sealing ring in the proximal direction.

It has been found that by using in total four sealing rings, a particularly good guidance can be provided and the tendency to tilt is reduced without the required forces for pushing the plunger notably increasing.

In the case of a preferred embodiment of the invention, the sealing rings have a rounded, in particular circular segment-shaped cross-section.

In particular, the sealing rings have a circular segment-shaped cross-section, which merges via a radius or a chamfer into the side wall.

The sealing rings pressed together thus, compared for example to lamellar sealing rings, form a relatively wide contact surface, which also improves the guidance.

A halogenated butyl rubber, in particular a chlorobutyl or a bromobutyl, is preferably used as the butyl rubber.

The internal thread of the plunger stopper is preferably formed as a double trapezoidal thread.

The thread teeth running relatively flat in such a thread cause a reduced expansion of the plunger stopper when it is pushed.

In the case of a further development of the invention, the plunger stopper comprises a coating at least on its side wall and/or end wall.

The side wall and the end wall are preferably provided with a coating.

In particular, the plunger stopper can comprise a coating of polytetrafluorethylene.

The coating can thereby serve to reduce the frictional force and form a migration barrier.

In the case of one embodiment of the invention, the ratio of a wall thickness of the side wall to the diameter d of the plunger stopper is more than 0.05, preferably more than 0.08 and particularly preferably more than 0.09 and/or less than 0.2, preferably less than 0.15, particularly preferably less than 0.11.

The wall thickness is also relatively thick even in syringes with a relatively small volume, in particular with a fill volume of less than 15 ml, which also serves for good guidance.

In the case of one embodiment of the invention, the first sealing ring and/or the further sealing rings have a height h of more than 0.3 mm, preferably more than 0.5 mm, particularly preferably more than 0.6 mm and/or less than 1.2 mm and/or preferably less than 0.9 mm, particularly preferably less than 0.8 mm.

It has been found that through such dimensioning in conjunction with a syringe body, which compresses the sealing rings, an optimal compromise can be found between sealing effect and necessary pushing force.

In the case of one embodiment of the invention, all sealing rings have the same height.

In the case of another embodiment of the invention, the sealing rings can also have at least in part a different height.

In particular, the distally first sealing ring can have a greater height than the subsequent sealing rings.

In this way, the sealing effect is improved in the region directly adjoining the medical fluid.

The sealing rings, which are between the proximally and distally outermost sealing rings, preferably have the same cross-section.

The sealing rings can have, in particular in an axial sectioned view, a substantially circular segment-shaped cross-section, wherein the circular segment forms an angle of over 90°, preferably over 120°. The circular segment preferably forms an angle of less than 180°, particularly preferably of less than 170°.

In the case of one embodiment of the invention, the proximal and/or distal flanks of at least one sealing ring merge over a radius, which is smaller than the radius of the sealing ring, into the side wall.

The proximal tip angle of the end wall preferably corresponds substantially to the distal tip angle of the end wall. The end wall thus has on the inside and outside the same tip angle and accordingly a roughly constant wall thickness.

The proximal tip angle and/or the distal tip angle is or are over 100°, preferably over 120°, particularly preferably over 140° and/or under 170°, preferably under 160° and particularly preferably under 155°.

The end wall of the syringe body preferably has a substantially corresponding shape in the case of a syringe equipped with the plunger stopper according to the invention. Thus, the syringe can be fully emptied.

In the case of one embodiment of the invention, the ratio of a tooth height z of the internal thread to a diameter d of the plunger stopper is over 0.04, preferably over 0.05 and/or under 0.15.

Thus, a sufficient secure connection is achieved between the thread of the plunger rod and the plunger stopper.

The diameter of the plunger stopper can be between 8 mm and 30 mm. The diameter of the plunger stopper is understood as its maximum diameter, i.e. in particular the diameter of the largest sealing ring.

The plunger stopper is in particular formed for syringes with a fill volume of 2.25 ml to 50 ml.

In the case of one embodiment of the invention, the wall thickness is 1 to 2 times, preferably 1.2 to 1.6 times, the wall thickness of the side wall.

The end wall can thus be formed somewhat thicker than the side wall, but still has a similar thickness in such manner that a simple manufacture of the plunger stopper is made possible.

The wall thickness of the side wall is understood as the wall thickness without the height of the thread teeth and without the height of the sealing rings.

These heights included, the thickness of the side wall in addition to thread and sealing ring are thus moved in a similar range as the end wall, which leads to a uniform cooling of the plunger stopper when it is manufactured in an injection-molding process.

The pitch of the internal thread is preferably between 0.4 and 0.8 mm, particularly preferably between 0.5 and 0.7 mm.

In the case of one embodiment of the invention, the length of the plunger stopper is between 8 mm and 16 mm, preferably between 10 mm and 14 mm.

The length is understood as the total length of the plunger stopper including the conical tip.

In the case of one embodiment of the invention, a proximal first sealing ring comprises a proximal flank which is flattened compared to the distal flank.

The sealing ring in particular comprises a circular segment-shaped cross-section, which merges on the distal flank into a tangent of the circle.

Such a configuration can lead to greater rigidity of the rear sealing ring during deformation.

The rear sealing ring thus runs virtually behind the subsequent sealing ring in the distal direction when pushed, which in turn reduces the tendency to tilt.

The invention also relates to a syringe with a plunger stopper, as has been described above.

In particular, the syringe comprises a plunger rod with a distal conical end, which rests on the conical inner side of the end wall of the plunger stopper.

The syringe is preferably filled with a medical fluid, in particular with a medical fluid containing a medication and sealed with a stopper. The stopper can comprise a tamper-evident seal.

The syringe can in particular be autoclaved. In particular, the syringe is located in an outer package and is autoclaved in the outer package.

The outer package can in particular be formed as a tear-open pouch made of foil.

### BRIEF DESCRIPTION OF THE DRAWINGS

The subject matter of the invention will be explained in more detail below with reference to exemplary embodiments represented on the basis of the drawings FIG. 1 to FIG. 7.

FIG. 1 is a sectioned view of a medical syringe according to the invention.

FIG. 2 is a sectioned view of an exemplary embodiment of a plunger stopper.

FIG. 3 is a sectioned view along the line A-A of FIG. 2.

FIG. 4 to FIG. 7 are further sectioned views of alternative exemplary embodiments of a plunger stopper.

### DETAILED DESCRIPTION OF THE DRAWINGS

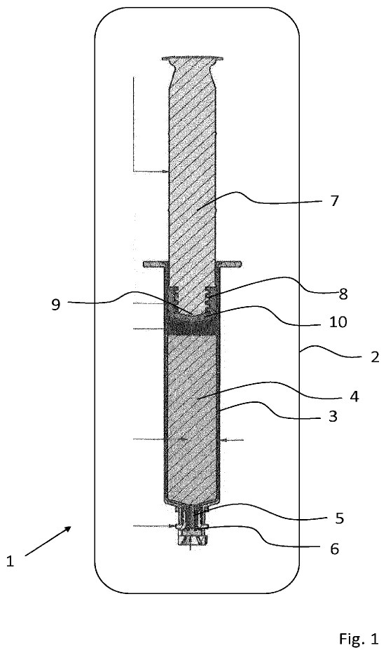

FIG. 1 shows, in a sectioned view, a medical syringe 1, which is formed with a plunger stopper according to the invention.

The syringe 1 is arranged here in the schematically represented outer package 2. The outer package 2 is formed as a tear-open foil package and the syringe 1 has been autoclaved in the outer package 2.

The syringe 1 comprises a syringe body 3, which provides a volume which is pre-filled with a medical fluid 4. The nozzle 5 of the syringe 1 is sealed with a stopper 6.

The inner volume of the syringe 1 can, in addition to the medical fluid 4, contain a gas, in particular air or nitrogen, in order to avoid deformations of the syringe body 3 in the case of pressure fluctuations.

In order to expel the medical fluid 4 after removing the stopper 6, the syringe 1 comprises a plunger rod 7 with a proximal gripping end, which is connected to the plunger stopper 10.

To this end, the plunger rod 7 comprises an external thread 8, which is screwed into the internal thread 16 of the plunger stopper 10.

The distal end 9 of the plunger rod 7 is conical, in this exemplary embodiment cone-shaped, and rests on the end wall 11 of the plunger stopper 10 correspondingly formed on the inside.

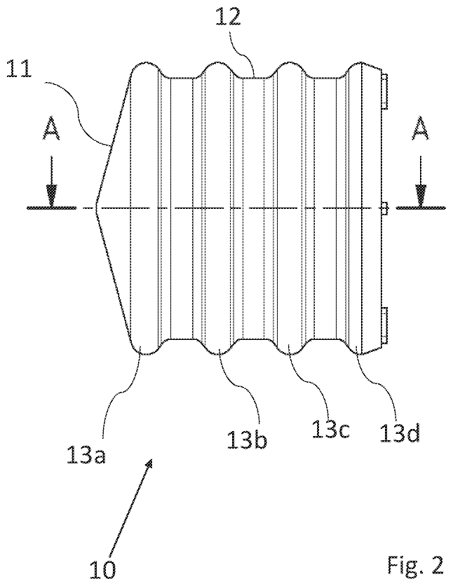

FIG. 2 is a plan view of the side wall 12 of a plunger stopper 10 according to the invention.

The outer contour of the plunger stopper 10 is rotationally-symmetric.

The plunger stopper 10 comprises a truncated cone-shaped end wall 11, wherein the cone of the end wall 11 merges seamlessly, i.e. without step, groove or similar, into a first sealing ring 13a.

The sealing rings 13b to 13d are adjoined distributed over the length of the plunger stopper 10, wherein the sealing ring 13d forms the proximal end of the plunger stopper 10.

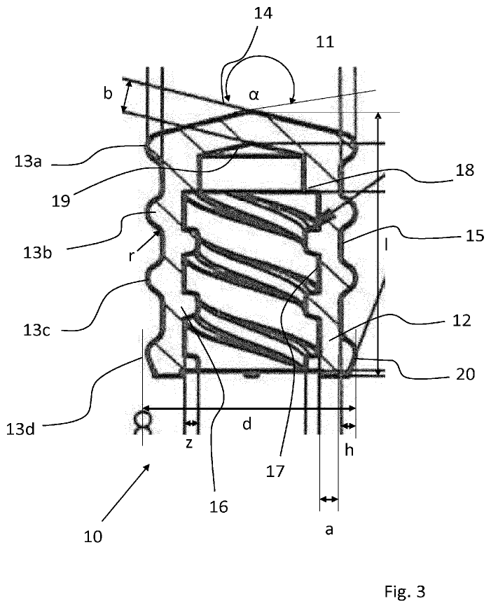

FIG. 3 is a sectioned view along the line A-A of FIG. 2, i.e. an axial section along the rotational axis of the plunger stopper 10.

The end wall 11 is a truncated cone shape and comprises the tip angle α, which can in particular be between 140° and 160°.

The end wall 11 is formed correspondingly in a truncated cone shape on the inner side 19. Consequently, a configuration of the end wall is formed which is roof-shaped in the cross-section.

The plunger stopper 10 is provided with an internal thread 16, which is formed as a two-pass trapezoidal thread.

The plunger stopper 10 has a (maximum) diameter d.

The internal thread 16 has teeth with a tooth height z. The teeth of the internal thread 16 can be directed forwards, in particular the distal wall of the teeth can form an angle of 2° to 10° to the transversal plane.

This ensures that the outer thread 8 of the plunger rod 7 does not slip out when the plunger is pulled back.

The wall thickness of the side wall a is by definition determined without the teeth of the thread 16 and without the height h of the sealing rings 13a to 13d.

The sealing rings 13a to 13d are formed monolithically with the rest of the plunger stopper.

The sealing rings 13a to 13c are in this exemplary embodiment formed in a substantially circular segment shape in the cross-section. The sealing rings 13a to 13d can have a radius of 0.6 to 0.7 in this exemplary embodiment.

The sealing rings 13a to 13d have a height h between 0.7 to 0.8 mm.

In this exemplary embodiment, the sealing rings 13a to 13d all have the same height.

The diameter d of the plunger stopper 10 is in this exemplary embodiment 9 to 9.5 mm.

The wall thickness of the side wall 12 is 0.9 to 1.0 mm.

The proximally last sealing ring 13d has a deviating shape compared to the other sealing rings 13a to 13c.

The contour, which has a circular segment shape in the cross-section, merges into a tangent on the proximal flank 20.

This tangent can in particular form an angle of 60° to 80° towards the side wall 12.

The length 1 of the plunger stopper can be between 10 and 14 mm.

In the case of this exemplary embodiment, the internal thread 16 emerges into the tip 14 of the plunger stopper 10 via a step 18, which is located between the distal sealing ring 13a and the subsequent sealing ring 13b.

In this transition section, the inner diameter of the plunger stopper 10 corresponds roughly to the diameter between the teeth of the thread 16.

In conjunction with a correspondingly shaped plunger rod 7, pushing the plunger stopper 10 means that tensions are concentrated at the tip 14 and the material of the plunger stopper 10 located behind follows after so to speak.

This achieves an optimal tilting security.

The wall thickness b of the end wall 11 is greater than the wall thickness of the side wall 12. This is in particular 1.2 to 1.8 times as great.

The outer side 15 of the side wall is formed straight between the sealing rings 13a to 13d. The side wall 12 thus has a circular cylindrical shape in the region between the sealing rings 13a to 13d.

The exemplary embodiment represented here is provided for syringes with a fill volume of 2.25 ml.

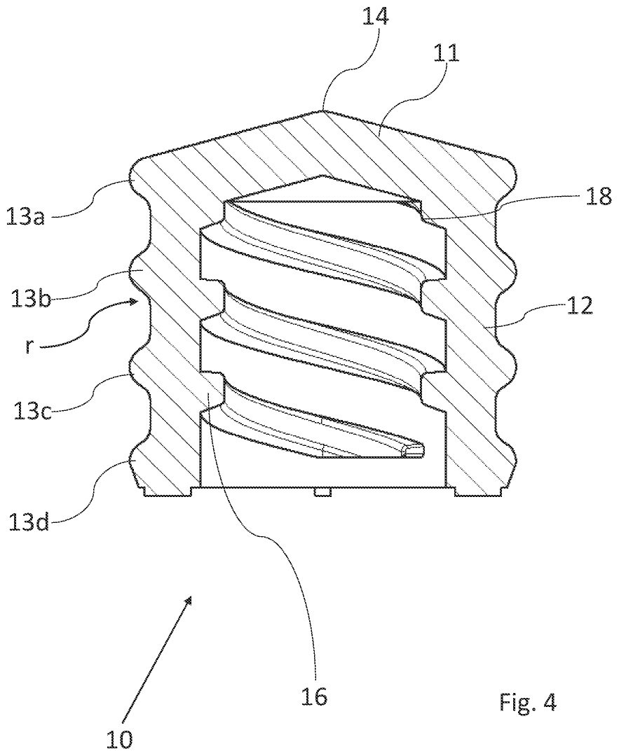

FIG. 4 to FIG. 7 are further corresponding sectioned views of exemplary embodiments of a plunger stopper 10, wherein the diameter of the plunger stopper is greater in each case.

The plunger stopper 10 represented in FIG. 4 thus has a greater diameter than the plunger stopper represented in FIG. 3, in particular a diameter d of between 12 and 13 mm.

The sealing rings 13a to 13c can have a contour corresponding to FIG. 3 and can in particular have the same dimensions.

Unlike the exemplary embodiment according to FIG. 3, the sealing rings 13a to 13d merge into the side wall 12 via a radius r. The radius r is smaller in this exemplary embodiment than the radius of the respective circular segment which forms the sealing ring 13 to 13d, in particular 0.5 to 0.9, preferably 0.7 to 0.8 times as great.

Side wall 12 and end wall 11 are formed thicker than in the example according to FIG. 3.

In this exemplary embodiment, the side wall 12 has a size or thickness a of 1.5 to 1.7 mm and the end wall 11 of 1.8 to 2.2 mm.

The teeth of the thread 16 are enlarged compared to the exemplary embodiment according to FIG. 3, by contrast preferably in accordance with the diameter of the plunger stopper 10. They can in particular have a height z of 0.7 to 0.8 mm.

The thread 16 also merges into the tip 14 via a step 18 in the plunger stopper 10 represented in FIG. 4.

The exemplary embodiment according to FIG. 4 is provided for syringes 1 with a fill volume of 5 ml.

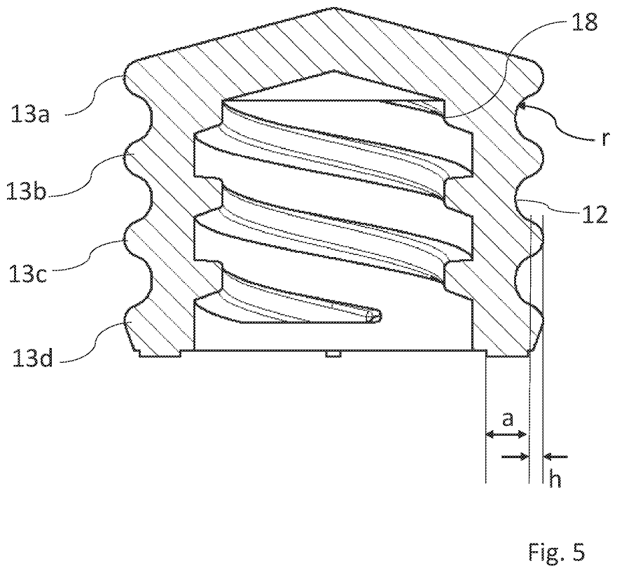

FIG. 5 shows an exemplary embodiment of a plunger stopper with a diameter d of 7.5 to 8.5 mm.

Unlike the embodiments represented in FIG. 3 and FIG. 4, the sealing rings 13a to 13d merge into rounded, in particular circular segment-shaped regions of the side wall 12.

The radius r of these regions of the side wall 12 can in particular be 1 to 1.5 times, preferably 1.1 to 1.3 times, the radius of the inner cross-section of the sealing rings 13a to 13d.

There is no circular cylindrical segments of the side wall located between the sealing rings 13a to 13b, but rather the intermediate spaces are rounded, in particular formed in a circular segment shape.

The radius of the inner cross-section of the sealing rings 13a to 13d can correspond to the previous exemplary embodiments.

The sealing rings 13a to 13d are in particular formed as in the case of the exemplary embodiments mentioned above.

By definition, the height h of the sealing rings 13a to 13d is understood as the height until reaching the turning point when transitioning to the radius r.

The thickness a of the side wall 12 is thus reduced by the radius r in each case.

The length 1 can also correspond to the exemplary embodiments FIG. 3 to FIG. 4.

The exemplary embodiment represented here is provided in particular for syringes with a fill volume of 10 ml.

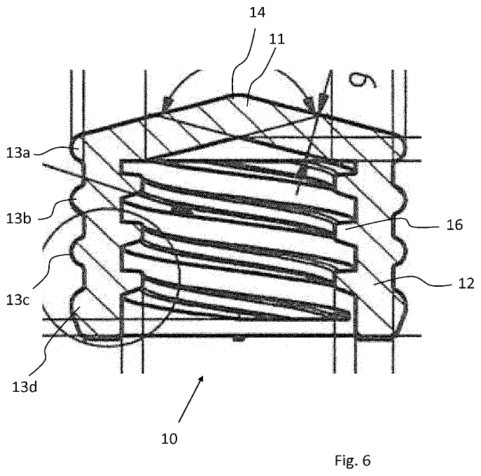

FIG. 6 shows an embodiment of a plunger stopper 10 with a diameter d of 19.5 to 20.5 mm.

The plunger stopper has a diameter d of 18 to 22 mm.

The inner radius of the cross-section of the sealing rings 13a to 13d is roughly greater than in the previous exemplary embodiments and can in particular be between 0.8 and 1.0 mm.

The side wall 12 is formed between the sealing rings 13a to 13d on the outside in turn in a circular cylindrical shape.

The wall thickness of the side wall 12 is between 1.8 and 2.7 mm.

The wall thickness of the end wall 11 is between 2.4 to 2.8 mm.

Furthermore, the thread 16 merges directly into the tip 14 in this exemplary embodiment.

The plunger stopper 10 represented here has a length of 12 to 15 mm.

The plunger stopper 10 can be provided according to this exemplary embodiment for a syringe 1 with a fill volume of 20 mm.

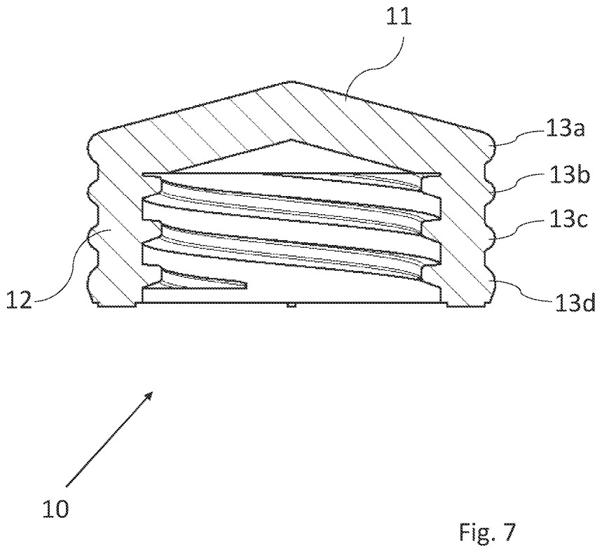

FIG. 7 shows a further sectioned view of an exemplary embodiment of a plunger stopper 10.

This plunger stopper has a diameter d of 25 to 29 mm and/or a length 1 of 11 to 15 mm.

In the case of this exemplary embodiment, the inner radius of the distally first sealing ring 13a is greater than that of the subsequent sealing rings 13b to 13d.

Thus, the inner radius of the first sealing ring 13a is 1.0 to 1.3 mm, whereas the inner radius of the cross-section of the sealing rings 13b to 13d is 0.8 to 0.9 mm.

The wall thickness of the side wall 12 is 2.8 to 3.2 mm. The wall thickness of the end wall 11 is 3.6 to 4 mm.

The teeth of the thread 16 have a height h between 1.1 to 1.5 mm.

The plunger stopper 10 represented in FIG. 7 is in particular formed for syringes 1 with a fill volume of 50 ml.

Through the invention, a plunger stopper 10 optimized for pre-filled medical syringes 1 can be provided which has only a very low tendency to tilt and provides good seal-tightness.

### LIST OF REFERENCE NUMERALS

α Tip angle
a Thickness of the side wall
b Thickness of the end wall
d Diameter of the plunger stopper
h Height of the sealing rings
1 Length of the plunger stopper
r Radius transition sealing ring to side wall
z Tooth height of the internal thread
1 Syringe
2 Outer package
3 Syringe body
4 Medical fluid
5 Nozzle
6 Stopper
7 Plunger rod
8 Thread
9 Distal end of the plunger rod
10 Plunger stopper
11 End wall
12 Side wall
13a First sealing ring
13b-13d Further sealing rings
14 Tip
15 Outer side of the outer wall
16 Thread
17 Thread base
18 Step
19 Inner side of the end wall
20 Proximal flank

1. A plunger stopper for a medical syringe, the plunger stopper comprising:
an end wall, a side wall, and an internal thread for securing a plunger rod,
wherein the plunger stopper is made of a butyl rubber,
wherein the end wall is conical in the distal direction both on the outside and on the inside,
wherein the end wall merges towards the side wall into a first sealing ring, which extends around the circumference of the side wall,
and wherein the side wall has at least three further sealing rings.

2. The plunger stopper according to claim 1, wherein the sealing rings have a circular segment-shaped, cross-section.

3. The plunger stopper according to claim 1, wherein the butyl rubber is a halogenated butyl rubber comprising a chlorobutyl or a bromobutyl.

4. The plunger stopper according to claim 1, wherein the internal thread is formed as a double trapezoidal thread.

5. The plunger stopper according to claim 1, further comprising a coating at least on the side wall and/or the end wall, the coating comprising a polytetrafluorethylene.

6. The plunger stopper according to claim 1, wherein a ratio of a wall thickness of the side wall to a diameter of the plunger stopper is more than 0.5 and/or less than 2.0.

7. The plunger stopper according to claim 1, wherein a proximal tip angle of the end wall corresponds substantially to a distal tip angle of the end wall.

8. The plunger stopper according to claim 1, wherein a ratio of a tooth height of the internal thread to a diameter of the plunger stopper is over 0.04 and/or under 0.15.

9. The plunger stopper according to claim 1, wherein a wall thickness of the end wall is 1 to 2 times the wall thickness of the side wall.

10. The plunger stopper according to claim 1, wherein a pitch of the internal thread is between 0.4 and 0.8 mm.

11. The plunger stopper according to claim 1, wherein a proximally first sealing ring has a proximal flank which is flattened compared to a distal flank.

12. A syringe having a plunger stopper according to claim 1.

13. The syringe according to claim 12, further comprising a plunger rod with a distal conical end, the distal conical end resting on the conical inside of the end wall.

14. The syringe according to claim 12, wherein the syringe is filled with a medical fluid and sealed with a stopper.

15. The syringe according to claim 12, wherein the syringe is autoclaved in an outer package in which the syringe is disposed.

16. The plunger stopper according to claim 6, wherein the ratio of the wall thickness of the side wall to the diameter of the plunger stopper is more than 0.9.

17. The plunger stopper according to claim 6, wherein the ratio of the wall thickness of the side wall to the diameter of the plunger stopper is less than 1.1.

18. The plunger stopper according to claim 8, wherein the ratio of the tooth height of the internal thread to the diameter of the plunger stopper is over 0.05.

19. The plunger stopper according to claim 1, wherein the wall thickness of the end wall is 1.2 to 1.6 times the wall thickness of the side wall.

20. The plunger stopper according to claim 10, wherein the pitch of the internal thread is between 0.5 and 0.7 mm.
